(19) United States
(12) Plant Patent Application Publication
(10) Pub. No.: US 2005/0210552 P1
(43) Pub. Date: Sep. 22, 2005

(54) Shrub rose plant names 'Poulaksel'

(76) Inventor: Mogens Olesen, Fredensborg (DK)

Correspondence Address: POULSEN ROSER APS, 620 S. FRONT STREET, CENTRAL POINT, OR, 97502, US

(21) Appl. No.: 11/125,599

(22) Filed: May 9, 2005

Related U.S. Application Data
(63) Continuation of application No. 10/647,013, filed on Aug. 21, 2003.

(51) Int. Cl. A01H 5/00

(52) U.S. Cl. PLT/104

(57) ABSTRACT

A new shrub rose plant which has abundant, yellow flowers and attractive foliage. This new and distinct variety has shown to be uniform and stable in the resulting generations from asexual propagation.

This application is a continuation of U.S. Plant patent application Ser. No. 10/647,013, dated Aug. 21, 2003.

### BOTANICAL CLASSIFICATION

Rosa hybrid

### VARIETY DENOMINATION

‘Poulaksel’

### SUMMARY OF THE INVENTION

The present invention constitutes a new and distinct variety of garden rose plant which originated from a controlled crossing between ‘Pouldiram’, described and illustrated in U.S. Plant Pat. No. 12,568 issued on 23 Apr., 2002 and an unnamed, non-patented seedling. The two parents were crossed during the summer of 1983 and the resulting seeds were planted in a controlled environment in Fredensborg, Denmark. The new variety is named ‘Poulaksel’.

The new variety may be distinguished from the seed parent, ‘Pouldiram’ by the following combination of characteristics:
1. The seed parent has a general tonality of White Group 155C, while that of ‘Poulaksel’ is Yellow Group 4B.
2. The seed parent has a petal count of 12 to 18, while that of ‘Poulaksel’ is 18 to 20.
3. The seed parent has a bloom diameter of 80 mm, while that of ‘Poulaksel’ is 60 mm.

The objective of the hybridization of this rose variety was to create a new and distinct variety for garden use with unique qualities, such as:
1. Uniform and abundant flowers;
2. Vigorous, but compact growth when propagated both as a budded rose and on its own roots;
3. Disease resistance.

This combination of qualities is not present in previously available commercial cultivars of this type and distinguish ‘Poulaksel’ from all other varieties of which we are aware.

As part of their rose development program, L. Pemille Olesen and Mogens N. Olesen germinated the seeds from the aforementioned hybridization during winter 1994 and conducted evaluations on the resulting seedlings in a controlled environment in Fredensborg, Denmark.

‘Poulaksel’ was selected in the spring 1995 by the inventors as a single plant from the progeny of the aforementioned hybridization.

Asexual reproduction of ‘Poulaksel’ by traditional budding and rooted cuttings was first done by L. Pemille and Mogens N. Olesen in their nursery in Fredensborg, Denmark in August, 1995. This initial and other subsequent asexual propagations conducted in controlled environments have demonstrated that the characteristics of ‘Poulaksel’ are true to type and are transmitted from one generation to the next.

### BRIEF DESCRIPTION OF THE DRAWING

The accompanying color illustration shows as true as is reasonably possible to obtain in color photographs of this type, the typical characteristics of the buds, flowers, leaves, and stems, of ‘Poulaksel’. Specifically illustrated in FIG. 1:

FIG. 1.1; Stem showing branching and the attachment of leaves, buds, and peduncles;

FIG. 1.2; Flower bud, partially opened bud, and open bloom;

FIG. 1.3; Flower petals, detached;

FIG. 1.4; Sepals, receptacle, and pedicel;

FIG. 1.5; Stem as well as a bare stem exhibiting thorns;

FIG. 1.6; Leaves.

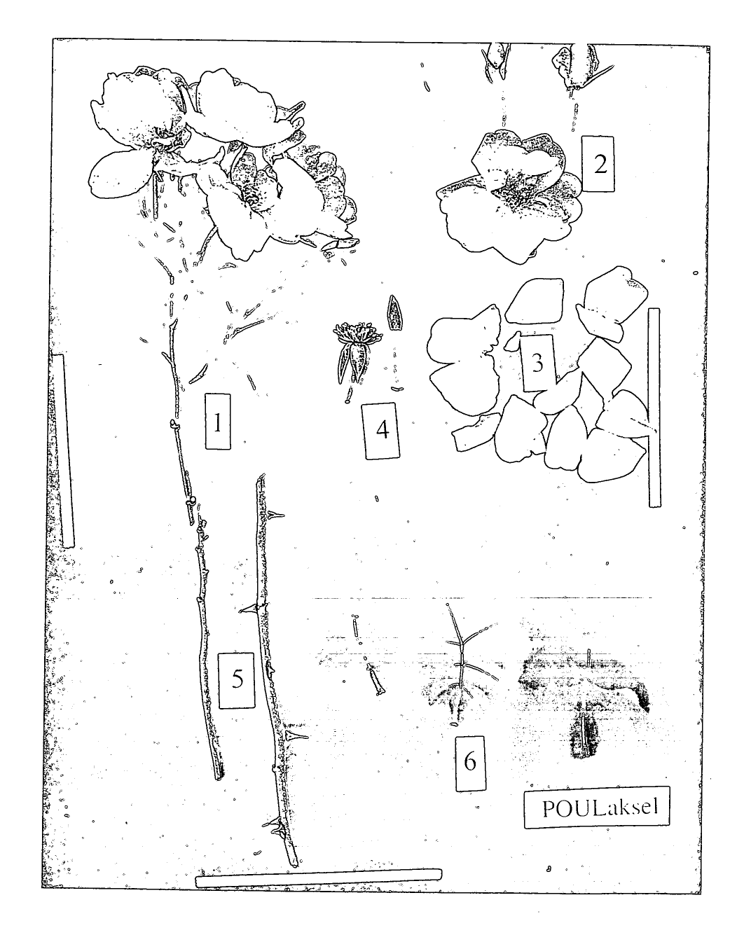

### DETAILED DESCRIPTION OF THE VARIETY

The following is a, description of ‘Poulaksel’, as observed in its growth in a field nursery in Jackson County, Oregon. Plants were grown for a period of 2 years on Rosa multiflora understock. Color references are made using The Royal Horticultural Society (London, England) Colour Chart, 1995, except where common terms of color are used.

For a comparison, several physical characteristics of the rose variety ‘Poulgode’, a rose variety from the same inventors described and illustrated in U.S. Plant patent application Ser. No. 10/782,360 dated 18 Feb., 2004 are compared to ‘Poulaksel’ in Chart 1.

| | ‘Poulaksel’ | ‘Poulgode’ |
|---|---|---|
| General tonality | Yellow Group 4B. | Yellow Group 8B, 9B |
| | | and 9C. |
| Bloom diameter | 60 mm. | 35 to 45 mm when open. |
| Petal count | 18 to 20. | 35 to 45. |

Parents:
   Seed parent.—‘Pouldiram’.
   Pollen parent.—unnamed seedling.

### FLOWER AND FLOWER BUD

Blooming habit: Continuous.
Flower bud:
   Size.—Upon opening, 27 mm in length from base of receptacle to end of bud.
   Bud form.—Elongate pointed ovoid.
   Bud color.—As sepals unfold, Yellow-Orange Group 14C, Yellow-Orange Group 14C at ¼ opening.
   Sepals.—Upper surface is Yellow-Green Group 144A at margins, Yellow-Green Group 144B in interior. Lower surface is Yellow-Green Group 144A at margins, Yellow-Green Group 144B in interior. Weak foliaceous appendages on three of the five sepals. Surfaces of sepals moderately pubescent. Stipitate glands are present along the margins of the sepals. Sepal apex is cirrose. Base is flat at union with receptacle. Sepals are generally 11 mm long×5 mm wide.
   Receptacle.—Texture: Smooth and lightly pubescent. Shape: Pear shaped. Size: Small. 6 mm (h)×5 mm (w). Color: Yellow-Green Group 146B.
   Peduncle.—Surface: With many small prickles and stipitate glands. Prickles are less than 1 mm long, colored Greyed-Purple Group 185C, and linear in shape. Length: 35 to 40 mm average length. Color: Yellow-Green Group 144B. Strength: Strong.
   Borne.—3 to 4 buds per flowering stem.
Flower bloom:
   Fragrance.—None.
   Duration.—The blooms have a duration on the plant of approximately 10 to 12 days.
   Size.—Average flower diameter is 60 mm when open.
   Form.—Flower viewed from the side: Upon opening, upper part: Flat. Upon opening, lower part: Convex. Open flower, upper part: Flat. Open flower, lower part: Convex.
   Petalage.—Very double. Average range: 18 to 20 petals under normal conditions with 4 to 6 petaloids.
Color:
   Upon opening, petals.—Outermost petals: Outer side: Yellow Group 4B. Inner Side: Yellow Group 4B. Innermost petals: Outer side: Yellow Group 4B. Inner Side: Yellow Group 4B.
   Upon opening, basal petal spots.—Outermost petals: Outer side: Yellow Group 5B. Inner Side: Yellow Group 5B. Innermost petals: Outer side: Yellow Group 5B. Inner Side: Yellow Group 5B.
   After opening, petals.—Outermost petals: Outer side: Marginal zone is Yellow Group 4C, middle zone is Yellow Group 5B. Inner Side: Marginal zone is Yellow Group 4C, middle zone is Yellow Group 5B. Innermost petals: Outer side: Marginal zone is Yellow Group 4C, middle zone is Yellow Group 5B. Inner Side: Marginal zone is Yellow Group 4C, middle zone is Yellow Group 5B.
   After opening, basal petal spots.—Outermost petals: Outer Side: Yellow Group 5B. Inner Side: Yellow Group 5B. Innermost petals: Outer Side: Yellow Group 5B. Inner Side: Yellow Group 5B.
General tonality: On open flower Yellow Group 4B. No change in the general tonality at the end of the 5th day. Afterwards, general tonality is Yellow Group 4D.
Petals:
   Petal reflex.—Strongly reflexed.
   Petal edge.—Entire.
   Shape.—Apex is rounded, base is acute.
   Petaloids.—Quantity: 4 to 6. Color: Upper surface is Yellow Group 4C and Yellow Group 5B; lower surface is Upper surface is Yellow Group 4C. Size: 12 mm long×10 mm wide.
   Thickness.—Average.
   Arrangement.—Not formal.
Reproductive organs:
   Pistils.—Length: 12 mm long. Quantity: 20 to 25.
   Pollen.—Quantity: None.
   Anthers.—Size: 1 mm long. Color: Interior is Greyed-Orange Group 166A. Margin is Greyed-Yellow Group 162B. Quantity: 35 to 40.
   Filaments.—Color: Yellow Group 13A. Length: 10 mm.
   Stigmas.—Roughly even in position relative to anthers. Color: Greyed-Yellow Group 160D.
   Styles.—Color: Yellow-Green Group 145C at base, White Group 155C in middle, graduating to Red-Purple Group 61C at top.
   Hips.—Shape: Round. Size: 13 to 15 mm in diameter. Color: Red-Purple Group 59B.

### PLANT

Plant growth: Vigorous and compact. When grown as a budded field grown plant on Rosa multiflora understock, the average height of the plant is 100 cm and the average width is 100 cm.
Stems:
   Color.—Young wood: Yellow-Green Group 144B. Older wood: Yellow-Green Group 144A.
   Thorns.—Incidence: 10 to 12 thorns per 10 cm of stem. Size: Average length: 9 mm. Color: Greyed-Purple Group 184B and C. Shape: Linear.
   Surface.—Young wood: Smooth. Older wood: Smooth.
Plant foliage: Normal number of leaflets on normal leaves in middle of the stem: 5 leaflets.
   Leaf size.—105 mm (l)×85 mm (w).
   Quantity.—Average.
   Color.—Mature Foliage: Upper Leaf Surface: Yellow-Green Group 146A. Lower Leaf Surface: Yellow-Green Group 147B. Juvenile foliage: Upper Leaf Surface: Green Group 137A. Lower Leaf Surface: Green Group 137C. Anthocyanin intonation: Location: New growth of top leaves, rachis, petiole and stipule. Color: Greyed-Purple Group 184B and C.
Plant leaves and leaflets:
   Stipules.—Size: 25 mm. Color: Yellow-Green Group 143A on upper surface, Yellow-Green Group 144A on lower surface. Margin: Stipitate glands present along the stipule margin.
   Petiole.—Length: 8 to 15 mm. Color: Yellow-Green Group 144A. Underneath: Yellow-Green Group 144A. Margins: Stipitate glands present. Anthocyanin: Present on juvenile petioles. Color is Red-Purple Group 59D.
   Rachis.—Color: Yellow-Green Group 144A. Underneath: Yellow-Green Group 144A. Margins: Stipitate glands present. Anthocyanin: Present on juvenile petioles. Color is Red-Purple Group 59D.
   Leaflet.—Edge: Serrated. Shape: Ovate. Glossiness: Glossy. Thickness: Thick. Arrangement: Odd pinnate. Venation: Reticulate. Size: 45 mm (l)×30 mm (w).
Disease resistance: Above average resistance to downy and powdery mildew, rust, black spot, and Botrytis under normal growing conditions in Jackson County, Oregon.
Cold hardiness: The variety ‘Poulaksel’ has been found to be cold hardy in to USDA cold hardiness zone 6.

1. A new and distinct variety of rose plant of the shrub rose class named ‘Poulaksel’, herein described and illustrated, due to its abundant Yellow Group 4B flowers, disease resistance, and extended period of bloom.
